(19) United States
(12) Patent Application Publication
(10) Pub. No.: US 2012/0178854 A1
(43) Pub. Date: Jul. 12, 2012

(54) IRON(III)-COMPLEXING AGENTS FOR STABILIZING COMB POLYMERS IN MINERAL BINDING AGENTS

(75) Inventors: Christian M. Burge, Schafisheim (CH); Andre Peter, Effretikon (CH); Christophe Kurz, Oberweningen (CH); Franz Wombacher, Jonen (CH)

(73) Assignee: SIKA TECHNOLOGY AG, BAAR (CH)

(21) Appl. No.: 13/395,805

(22) PCT Filed: Sep. 21, 2010
(86) PCT No.: PCT/EP2010/063907

§ 371 Date: Mar. 13, 2012

(30) Foreign Application Priority Data
Sep. 21, 2009 (EP) 09170837.0
Sep. 22, 2009 (EP) 09170917.0

(51) Int. Cl. C08K 3/00; C07C 331/08; C08K 5/17; C08K 5/47; C08K 5/37; C07D 277/40; C07C 229/26

(52) U.S. Cl. 524/2; 548/199; 558/13; 562/566; 524/83; 524/208; 524/239

(57) ABSTRACT

Compositions including a comb polymer and an Fe(III)-complexing agent X selected from the group consisting of thiocyanates, condensed phosphates, amines, amino acetic acids, oxycarboxylic acids and compounds of the formula (V). The invention also relates to a method for stabilizing comb polymers in the presence of Fe(III) using such Fe(III)-complexing agents X.

### TECHNICAL AREA

The invention relates to the area of additives for hydraulically setting compositions.

### PRIOR ART

It has been known for a long time that the raw materials used in cement production can have a more or less high chromium content. In particular the presence of water-soluble chromium (VI) in cement powder may lead, after longer skin contact, to skin rashes, in particular the so-called bricklayer's eczema, and is for this reason undesirable. Also known are methods and measures for reducing the undesirable soluble chromium (VI) content and fix it at the reduced level. For this purpose for example iron (II)-sulfate is used, whereby the water-soluble chromate fraction in cements are reduced to trivalent chromium by iron(II)-sulfate, and iron(II)-ions are oxidized to iron (III)-ions.

However, admixtures of known iron(II)-compounds to dry cement powder may lose their reducing effect frequently already after a relatively short time through oxidation by means of atmospheric oxygen of iron(II)-sulfate to iron(III)-compounds, because they can oxidized to inert iron(III)-compounds already with air intake. A particular disadvantage of iron (III)-compounds is that they are able to damage organic compounds present in the cement, in particular comb polymers, for example by an iron(III)-catalyzed oxidation of the organic compound with atmospheric oxygen.

For these reasons iron(II)-compound must be added in considerable amounts, typically 0.4% by volume of iron(II)-compound relative to the cement in order to ensure a safe chromium reduction. This leads therefore to a considerable proportion of potential iron(III)-compounds.

Comb polymers have been in use already for quite some time in concrete technology as dispersing agents, in particular as high-speed condensers because of their strong water reduction properties.

Comb polymers are only stable at higher temperatures under certain conditions and are destroyed within days, so that they are no longer able to have any effect. In particular it has been shown that the effectiveness of comb polymers decreases substantially if used in conjunction with iron(III)-compounds at higher temperatures in the presence of inorganic powders, in particular hydraulic binding agents. Such problems arise for example with the storage of hydraulic binding agents or when grinding hydraulic binding agents.

During storage hydraulic binding agents are usually kept in silos at over 80° C., often even at over 120° C. In addition, high pressures may occur in the silos, in particular in tower silos, making it difficult to stabilize the polymers. If comb polymers are added to the binding agent before, which is desirable in particular with the production of ready mixes such as cement ready-mixes, the effectiveness of the comb polymers decreases sharply after storage at high temperatures.

Comb polymers are to some extent also used as grinding agents when grinding hydraulic binding agents, for example clinkers, as for example described in WO 2005/123621A1. Since grinding also generates high temperatures, most comb polymers may be destroyed and will be no longer effective.

### EXPLANATION OF THE INVENTION

The purpose of the present invention is therefore to provide a composition and a method that protects comb polymers in the presence of iron(III)-compounds from damage caused by iron(III)-compounds, in particular in mineral binding agents and in particular at higher temperatures.

Surprisingly it was found that this can be achieved by a composition according to claim 1. It was noted that comb polymers could be protected from damage caused by iron(III)-compounds by compositions comprising a comb polymer and at least one Fe(III)-complexing agent X selected from a group consisting of thiocyanates, condensed phosphates, amines, amino acetic acids, oxycarboxylic acids and compounds of the formula (V), and which are able to maintain their effectiveness over longer periods of time in the presence of iron(III)-compounds even at higher temperatures.

It is particularly surprising that a composition comprising a comb polymer and at least one Fe(III)-complexing agent X, and which has in addition an antioxidant, is particularly suitable for protecting comb polymers from damage caused by iron(III)-compounds.

Furthermore, it became evident that comb polymers protected in this way are able to maintain their effectiveness as grinding agents and/or dispersing agents, in particular as condensers, even after having been exposed to high temperatures during the grinding process or after long storage.

Other aspects of the invention are the subject of further independent claims. Particularly preferential embodiments of the invention are the subject of the dependent claims.

### METHOD FOR EXECUTING THE INVENTION

The present invention relates to a composition comprising a comb polymer and at least one Fe(III)-complexing agent X selected from the group consisting of thiocyanates, condensed phosphates, amines, amino acetic acids, oxycarboxylic acid and compounds of the formula (V).

The term “Fe(III)-complexing agent” refers in this entire document to a neutral molecule and/or ion that is suitable to attach itself as a so-called ligand to an Fe(III), the so-called central ion, and form a complex compound as described in CD Römpp's Encyclopedia of Chemistry, 9^{th} edition, version 1.0, George Thieme Publisher, Stuttgart.

It is of advantage if the Fe(III)-complexing agent X has a complexing constant for Fe(III) which is 10 times, in particular 1000 times larger than the complexing constant for Fe(II). This is particularly advantageous if complexing Fe(III)-complexing agent X with Fe(II) is detrimental.

The term “complexing constant” refers in this entire document to the equilibrium constant K₁ for the formation of a complexing compound consisting of Fe(III) or Fe(II) respectively, (central ion) and an Fe(III)-complexing agent X (ligand), measured in water at ambient temperature.

2-Nitro-4-thiocyanatoaniline and benzyl thiocyanate, in particular benzyl thiocyanate, are preferred as thiocyanates.

Pyrophosphate, tripolyphosphate and polyphosphate are preferred as if condensed phosphates.

Amines and amino acetic acids are advantageous because they are able to enter into very stable complexing compounds with Fe(III).

Triethanolamine (TEA), ethylendiamine (EDA), 1-[2-(bis(2-hydroxypropyl)amino)ethyl(2-hydroxypropyl)amino]propan-2-ol (quadrol), N,N-di-(hydroxyethyl)-glycine (DHEG), diethylentriamine (DETA), tetraethylenpentamine (TEPA), triethylentetramine (TETA) are preferred as amines.

Nitrilotri acetic acid (NTA), ethylendiamine tetraacetic acid (EDTA), cyclohexanediaminetetraacetic acid (CDTA), diethylentriaminpentaacetic acid (DTPA), ethyleneglycol-bis(aminoethylether)-N,N,N′N′ tetraacetic acid (EGTA), N-(2-hydroxyethyl)-ethylendiamine-N,N,N′ triacetic acid (HEDTA), triethylenetetraminehexaacetic acid (TTHA), glycine, glutaminic acid, and/or their salts are preferred as amino acetic acids.

Glycine has a stability constant for Fe(III) of 6.3×10¹⁰ and for Fe(II) of 2×10⁴. Glutamate has a stability constant for Fe(III) of 6.3×10¹³ and for Fe(II) of 4×10⁴. NTA has a stability constant for Fe(III) of 8×10¹⁵. EDTA has a stability constant for Fe(III) of 1.3×10²⁵ and for Fe(II) of 2×10¹⁴.

2,3-dihydroxybutanedioic acid, 2-hydroxy-1,2,3-propanetricarboxylic acid (citric acid), gluconic acid, α-hydroxybutyric acid, hydroxysuccinic acid are preferred as oxycarboxylic acids.

Glycolates have a stability constant for Fe(III) of 5×10³ and for Fe(II) of 8×10¹. Malonates have a stability constant for Fe(III) of 2×10⁹ and for Fe(II) of 5×10⁵. Citrate has a stability constant for Fe(III) of 3.2×10¹³ and for Fe(II) of 5×10⁵. Tartaric acid has a stability constant for Fe(III) of 7.2×10¹¹.

For compounds of the formula (V)

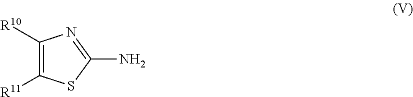

R¹⁰ and R¹¹ stand independently of each other for H, halogen atom, NO₂ or for one possibly branched, saturated or unsaturated alkyl radical with 1 to 25 C-atoms or an aryl radical with 6 to 14 C-atoms or an acyl radical with 1 to 10 C-atoms or a carbonyl radical with 1 to 10 C-atoms or a carboxyl radical with 1 to 10 C-atoms, or

R¹⁰ and R¹¹ are part of an aromatic or cyclo-aliphatic ring, in particular an aromatic 6-ring.

Examples of compounds of the formula (V) are 2-amino-5-bromothiazole, 2-amino-5-chlorothiazole, 2-amino-5-nitrothiazole, 2-aminothiazole, 2-aminothiazole-5-carboxaldehyde, 2-amino-4-thiazole carboxylic acid, 2-amino-5-methylthiazole, 2-amino-4-(trifluoromethyl) thiazole-5-carboxylic acid, 2-amino-4-thiazole acetic acid, 2-amino-4,5-dimethylthiazole, 2-amino-α-(methoxyimino)-4-thiazole acetic acid, 5-acetyl-2-amino-4-methylthiazole, 5-acetyl-2-amino-4-methylthiazole, methyl-2-amino-4-thiazolacetate, methyl-2-amino-4-thiazolacetate, 2-amino-4,6-difluorobenzothiazole, 2-amino-5-bromobenzothiazoel, 2-amino-6-bromobenzothiazole, 2-amino-4-chlorobenzothiazole, 2-amino-6-chlorobenzothiazole, 2-amino-6-fluorobenzothiazole, 2-amino-6-nitrobenzothiazole, 2-aminobenzothiazole, ethyl-2-amino-α-(hydroxyimino)-4-thiazolacetate, ethyl-2-amino-4-methylthiazole-5-carboxylate, ethyl-2-aminothiazole-4-acetate, ethyl-2-aminothiazole-4-acetate, 2-amino-4,5,6,7-tetrahydrobenzothiazole, 2-amino-6-(trifluoromethyl)benzothiazole, 2-amino-6-thiocyanatobenzothiazole, 2-amino-4-methoxybenzothiazole, 2-amino-6-methoxybenzothiazole, 2-amino-4-methylbenzothiazole, 2-amino-6-methylbenzothiazole, 2-amino-4-(3,4-difluorophenyl)thiazole, 2-amino-4-(4-bromophenyl)thiazole, 2-amino-4-(4-chlorophenyl)thiazole, 2-amino-4-(4-nitrophenyl)thiazole, 2-amino-4-phenylthiazole, 2-amino-4-phenylthiazole, 2-amino-6-ethoxybenzothiazole, 2-amino-5,6-dimethylbenzothiazole, 2-amino-4(p-tolyl)thiazole, 2-amino-5-methyl-4-phenylthiazole, 4-(4-acetamidophenyl)-2-aminothiazole, ethyl-2-amino-4-(4-bromophenyl)thiazole-5-carboxylate, ethyl-2-amino-4-phenylthiazole-5-carboxylate, 2-amino-4-phenyl-5-tetradecylthiazole.

2-aminothiazole is particularly preferred.

Preferably the Fe(III)-complexing agent X is a compound of the formula (V).

It is also particularly advantageous if the Fe(III)-complexing agent X is not made up of materials that lead to odor problems when used in mineral binding agents.

It is of particular advantage if the Fe(III)-complexing agent X has the effect of reducing Fe(III) to Fe(II).

The iron(II)-compound for reducing soluble chromium(VI) can be added to cement either as a cement additive during cement production or can be used as a concrete additive during concrete production. When used as a grinding additive, the dosage can be adjusted to the soluble chromium(VI) content. Usually 0.4% by volume (relative to the cement) iron(II)-sulfate is added so that the manufactured cements fulfill the requirements of Directive 2003/53/EC. When used as a concrete additive in concrete production, the added amount of the iron(II)-compounds cannot be adjusted to the chromium(VI) content of the cement since the chromium(VI) content of the cement is usually not known. In Germany the chromium(VI) content may amount to as much as 2 mg/kg.

Iron(II)-sulfate is usually used as iron(II) compound, but other iron(II) compounds, in particular iron(II) salts are also possible.

The iron(III) ions typically result from the oxidation of iron(II) ions of the mentioned iron(II) compound, in particular by atmospheric oxygen or chromium(VI) ions. Iron(III) ions may however also have a different origin. Iron(III) ions may thus for example come from iron(III) compounds, in particular iron(III) salts.

The term “comb polymer” in this present document refers to a comb polymer consisting of a linear polymer chain (=main chain) to which side chains are bonded via ester or ether groups. The side chains form here so to say the “teeth” of a “comb”.

The at least one comb polymer is preferably a comb polymer KP with side chains bonded to the main chain via ester or ether groups.

Suitable as comb polymer KP are on the one hand comb polymers with side chains bonded to the linear polymer frame via ether groups.

Side chains bonded to the linear polymer frame via ether groups can be inserted by polymerization of vinyl ethers or allyl ethers.

Such comb polymers are for example disclosed in WO 2006/133933 A2, the content of which is herewith included specifically by reference. The vinyl ethers or allyl ethers have in particular the formula (II).

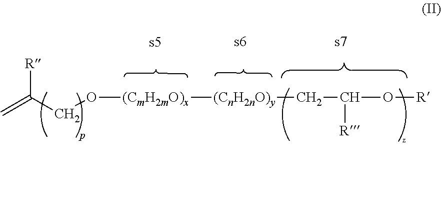

where R′ stands here for H or for an aliphatic hydrocarbon radical with 1 to 20 C-atoms or a cyclo-aliphatic hydrocarbon radical with 5 to 8 C-atoms or a possibly substituted aryl radical with 6 to 14 C-atoms. R″ stands for H or for a methyl group and R″ stands for an unsubstituted or substituted aryl radical, in particular for a phenyl radical.

Also, p stand for 0 or 1; m and n independently of each other for 2, 3 or 4 respectively; and x and y and z stand, independently from each other, for values ranging from 0 to 350.

The sequence of the partial structure elements described in formula (II) as s5, s6 and s7 can be arranged in this case as alternating, block-like or at random.

In particular such comb polymers are copolymers of vinyl ether or allyl ether with maleic acid anhydride, maleic acid, and/or (meth)acrylic acid.

Suitable as comb polymer KP on the other hand are comb polymers with side chains bonded to the linear polymer frame via ester groups. This type of comb polymers KP is preferred over the comb polymers bonded to the linear polymer frame via ether groups.

Particularly preferred comb polymers KP are copolymers of the formula (I).

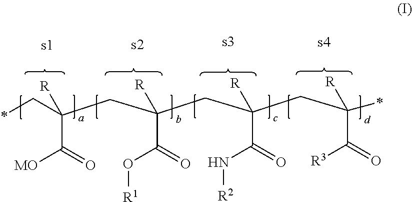

where M stand independently from each other for H⁺, alkali metal ion, alkaline earth metal ion, bi- or trivalent metal ion, ammonium ion, or organic ammonium group. The term “independently from each other” in this document means in each case that a substituent may have different available expressions in the same molecule. Thus for example the copolymer of the formula (I) may at the same time have carboxylic acid groups and sodium carboxylate groups, i.e. in this case H⁺ and Na⁺ have a different expression for M independently from each other.

The person skilled in the art understands that it is question on the one hand of a carboxylate to which the ion M is bonded, and that on the other hand in case of multi-valent ions M the charge must be balanced by counterions.

Also, the substituents R stand independently from each other for hydrogen or for a methyl group.

Moreover, the substituents R¹ stand independently from each other for -[AO]_{q}—R⁴. The substituents R² stand independently from each other for a C₁ to C₂₀ alkyl group, cycloalkyl group, alkylaryl group or for -[AO]_{q}—R⁴. The substituent A stands in both cases independently from each other for a C₂ to C₄ alkyl group and R⁴ for a C₁ to C₂₀ alkyl group, cyclohexyl group or alkylaryl group, while q represents a value from 2 to 250, in particular from 8 to 200, with particular preference from 11 to 150.

Also, the substituents R³ stand independently from each other for —NH₂, —NR⁵R⁶, —OR⁷NR⁸R⁹. Here R⁵ and R⁶ stand independently from each other for a C₁ to C₂₀ alkyl group, cycloalkyl group or alkylaryl group or aryl group or for a hydroxyalkyl group or for an acetoxyethyl-(CH₃—CO—O—CH₂—CH₂) or a hydroxyl-isopropyl-(HO—CH(CH₃)—CH₂) or an acetoxyisopropyl group (CH₃—CO—O—CH(CH₃)—CH₂); or R⁵ and R⁶ form together a ring of which nitrogen is a part, in order to build up a morpholine or a imidazoline ring.

The substituent R⁷ stands for a C₂-C₄ alkylene group.

Furthermore, the substituents R⁸ and R⁹ stand, independently from each other, for a C₁ to C₂₀ alkyl group, cycloalkyl group, alkylaryl group, aryl group or for a hydroxyalkyl group.

The sequence of the partial structure elements described in formula (I) as s1, s2, s3 and s4 can be arranged in this case as alternating, block-like or at random.

Finally the indices a, b, c and d represent the molar ratios of the structural units s1, s2, s3 and s4. These structural components have the following relationship to each other,
a/b/c/d=(0.1−0.9)/(0.1−0.9)/(0−0.8)/(0−0.3),

in particular a/b/c/d=(0.1−0.9)/(0.1−0.9)/(0−0.5)/(0−0.1),

preferably a/b/c/d=(0.1−0.9)/(0.1−0.9)/(0−0.3)/(0−0.06),

under the condition that a+b+c+d=1. The sum c+d is preferably larger than 0.

The production of the comb polymers KP of the formula (I) may take place, on the one hand, by a radical polymerization of the respective monomers of the formulas (III)ₐ, (III)_{b}, (III)_{c} and/or (III)_{d}, which then lead to the structural components structural units s1, s2, s3 and s4,

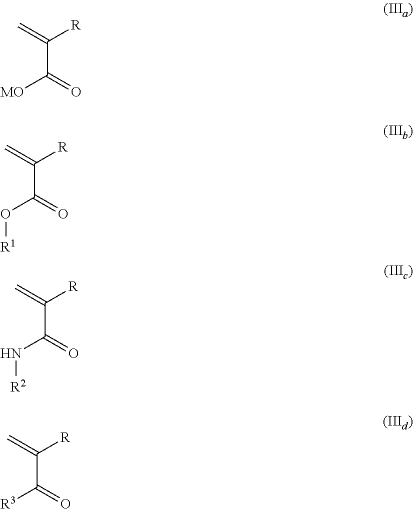

or, on the other hand, by a so-called polymer-analog transformation of a polycarboxylic acid of the formula (IV)

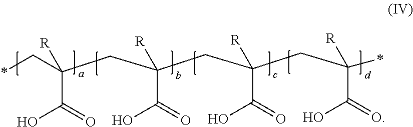

In a polymer-analog transformation the polycarboxylic acid of the formula (IV) is esterified or amidated with the respective alcohols or amines and then if necessary neutralized or partially neutralized (depending on the type of the radical M using e.g. metal hydroxides or ammonia). Details of the polymer-analog transformation are disclosed for example in US 2002/0002218 A1 on page 5 in section [0077] up to and including [0083] as well as in its examples, or in U.S. Pat. No. 6,387,176 B1 on page 5, line 18 to line 58 as well as in its examples. In a modification thereof, as described in US 2006/0004148 A1 on page 1 section [0011] up to and including [0055] on page 3 as well as in its examples, the comb polymer KP of the formula (I) can be produced in a solid aggregate state. The disclosure of the just mentioned patents is hereby specifically included by reference.

It turned out that particularly preferred embodiments of the comb polymers KP of formula (I) are those in which c+d>0, in particular d>0. —NH—CH₂—CH₂—OH has proven particularly advantageous as radical R³.

Particularly advantageous comb polymers KP proved to be those sold commercially by Sika Schweiz AG under the trade name series ViscoCrete®.

Typically the part by weight of the Fe(III)-complexing agent X amounts to 0.01 to 50% by weight, preferably 0.05 to 20% by weight, with special preference for 0.1 to 5% by weight relative to the total weight of the comb polymer.

It is also advantageous for the composition to have furthermore at least one antioxidant, in particular in the amount between 0.01 and 50% by weight, preferably 0.05 to 10% by weight, with particular preference for 0.1 to 5% by weight relative to the total weight of the comb polymer.

Suitable antioxidants are for example selected from the group consisting of substituted phenols, in particular sterically hindered phenols; substituted hydroquinones, in particular sterically hindered hydroquinones; sterically hindered aromatic amines such as diarylamine; arylamine-ketone-condensation products; organosulfuric compounds such as dialkyldithiocarbamine acids or dialkyldithiophosphites; organophosphorus compounds such as phosphites or phosphonites; tocopherols and their derivatives; gallic acids and their derivatives and vanillin.

Antioxidants comprising at least one substituted phenol, a substituted hydroquinone or a substituted aromatic amine are particularly suitable. Also very suitable are sterically hindered phenols, sterically hindered hydroquinones or sterically hindered aromatic amines.

Examples of sterically hindered phenols are 2-t-butyl-4,6-dimethylphenol, 2,6-di(t-butyl)-4-methyl phenol (butylhydroxytoluene, BHT), 2-t-butyl-4-methoxyphenol (butylhydroxyanisol, BHA), pentaerythrityl-tetrakis-[3 (3,5-ditert.-butyl-4-hydroxyphenyl)-propionate] (Irganox® 1010), 2,6-dioctadecyl-4-methylphenol, 2,4,6-tri-t-butylphenol, ortho-tert-butylphenol, 3,5-bis(1,1-dimethylethyl)-4-hydroxybenzene-propionic acid ester of C4-C22 alcohols, 4,4′-butyliden-bis-(6-t-butyl-3-methylphenol), 4,4′-methyliden-bis-(2,6-di-t-butylphenol), 3,5-bis(1,1-ditert.-butyl)-4-hydroxyphenyl-propionic acid ester of C4-C22 alcohols, 2,2′-methylene-bis-(6-tert-butyl-4-methylphenol), 2,2-bis-(4-hydroxyphenyl)-propane(bisphenol A), 4,4′-methylenebisphenol (bisphenol F), 1,1-bis-(5-t-butyl-4-hydroxy-2-methylphenyl)-butane, 2,2′-methylene-bis-[4-methyl-6-(1-methylcyclohexyl)-phenol], tetrakis[methylene-3-(3,5-di-t-butyl-4-hydroxyphenyl)-propionate]methane, N,N′-hexamethylene-bis (3,5-di-t-butyl-4-hydroxy-hydrocinnamate amide), octadecyl-3-(3,5-di-t-butyl-4-hydroxyphenyl)-propionate, 1,3,5-tris-(3,5-di-t-butyl-4-hydroxybenzyl)isocyanurate, 1,1,3-tris(5-t-butyl-4-hydroxy-2-methylphenyl)-butane, 1,3,5-tris(3,5-di-t-butyl-4-hydroxybenzyl)mesitylene, ethylene glycol-bis[3,3-bis(3′-t-butyl-4′-hydroxyphenyl)butyrate], di-(3-t-butyl-4′-hydroxy-5-methylphenyl)dicyclopentadiene, 2.2′-methylene-bis-(4-methyl-6-cyclohexyl-phenol), 1,3,5-tri-(3,5-di-t-butyl-4-hydroxybenzyl)-2,4,6-trimethylbenzene (Irganox® 1330), 1,3,5-tris-(4-t-butyl-3-hydroxy-2,6-dimethylbenzyl)-isocyanurate, 3,5-di-t-butyl-4-hydroxyphenyl-propionic acid ester, 5-t-butyl-4-hydroxy-3-methylphenyl-propionic acid ester, 3,5-di-t-butyl-4-hydroxyphenyl-propionic acid amide, 3,5-di-(1,1-dimethylethyl)-4-hydroxybenzolpropionic acid ester, 1,6-hexanediol-bis-(3,5-di-t-butyl-4-hydroxyphenyl)propionate, triethylene glycol-bis-3-(t-butyl-4-hydroxy-5-methylphenyl)-propionate, 2,2-bis-(4-hydroxyphenyl)-propane, 2.2′-thio-bis-(4-methyl-6-t-butylphenol), 2-methyl-4,6-bis-((octylthio)-methyl)phenol (Irganox® 1520), 4.4′-thio-bis-(6-t-butyl-3-methylphenol), 2.2′-thiodiethyl-bis-3-(3,5-di-t-butyl-4-hydroxyphenyl)propionate.

Preferred are 2,6-di-t-butyl-4-methylphenol (BHT), octadecyl-3-(3,5-di-t butyl-4-hydroxyphenyl)propionate, 2,2′-methylene-bis-(4-methyl-6-cyclohexyl-phenol), 2,2′-methylene-bis-(4-methyl-6-t-butylphenol), triethylene glycol-bis-3-(t-butyl-4-hydroxy-5-methylphenyl)-propionate, 2,2-bis-(4-hydroxyphenyl)-propane and tetrakis[methylene-3-(3,5-di-t-butyl-4-hydroxyphenyl)-propionate] methane, or antioxidants as they are typically sold under the trademark Irganox® by the company Ciba Spezialitätenchemie, in particular 2-methyl-4,6-bis-((octylthio)-methyl)phenol (Irganox® 1520), pentaerythrityl-tetrakis-[3-(3,5-ditert.-butyl-4-hydroxyphenyl)-propionate] (Irganox® 1010), or 1,3,5-tri-(3,5-di-t-butyl-4-hydroxybenzyl)-2,4,6-trimethylbenzene (Irganox® 1330).

Examples for sterically hindered hydroquinones are 2,6-di-t-butyl-4-methoxyphenol, 2,5-di-t-butyl-hydroquinone.

Examples of sterically hindered aromatic amines and arylamine ketone condensation products are N,N′-bis-(1,4-dimethyl-pentyl)-p-phenylene-diamine, N,N′-diphenyl-p-phenylendiamine, 4-(p-toluene-sulfonamido)-diphenylamine, 4-n-butylaminophenol, 4,4′-di-t-octyldiphenylamine, 4,4′-di-(alpha, alpha dimethylbenzyl)-diphenylamine, phenyl-beta-naphthylamine, N-isopropyl-N′-phenyl-p-phenylendiamine, and/or phenyl-2-aminonaphthaline.

Examples of organosulfuric compounds are 2,2′-thio-bis-(4-methyl-6-t-butylphenol), 2-methyl-4,6-bis((octylthio)-methyl)phenol (Irganox® 1520), 4,4′-thio-bis-(6-t-butyl-3-methylphenol), 2,2′-thiodiethyl-bis-3-(3,5-di-t-butyl-4-hydroxyphenyl)propionate, 2,4-bis-(n-octylthio)-6-(4-hydroxy-3,5-di-t-butylanilino)-1,3,5-triazine, di-lauryl-3,3′-thiodipropionate, di-stearyl-3,3′-thiodipropionate, sodium dithionite, toluene sulfinic acid or their derivatives, for example sodium hydroxymethane sulfinate dihydrate (Rongalit® C).

Examples of organophosphorus compounds are 3,5-di-t-butyl-4-hydroxybenzyl-phosphonic acid-dioctadecylester, trimethylphosphite, triethylphosphite, triphenylphosphite, distearylpentaerythritdiphosphite, trichloroethylene-(nonylphenyl)-phosphite, tetrakis-(2,4-di-t-butylphenyl-4,4′-biphenylendiphosphonite, trichloroethylene-(2,4-di-t-butylphenyl)phosphite, neopentylglycoltriethylene glycoldiphosphite, diisodecylpentaerythritdiphosphite, tristearylphosphite, trilaurylphosphite, Na-hypophosphite or triphenylphosphite.

In a preferred embodiment the antioxidant is a substituted phenol, in particular a sterically hindered phenol. Such antioxidants are for example available under the name Irganox® from Ciba. Preferably the substituted phenol is selected from the group consisting of butylhydroxytoluene (BHT), butylhydroxyanisole (BHA), bisphenol A, bisphenol F, salicylic acid, hydroquinone, vanillin, biphenyldiol, for example 4, 4′-biphenyldiol or 2,2′-biphenyldiol, gallates and phenol poly condensation products.

The composition as described above can be used both in liquid and in solid form, both alone or as an integral part of a further composition, as dispersing agent, in particular as condenser; as a grinding agent, as a thickener or as cement refiner.

The composition as described above may include other components. Examples of other components are solvents or additives such as grinding agents, for example glycols or alkanolamines such as triisopropanolamine (TIPA) or triethanolamine (TEA); condensers, for example lignosulfonate, sulfonated naphthaline-formaldehyde condensates, or sulfonated melamine-formaldehyde condensates; accelerators; retardants; shrinkage reducers; antifoaming agents; foamers or components that reduce the separation of fresh concrete, in particular the discharge of water (bleed), and improve the agglomerative capacity of fresh concrete.

If the composition is used in liquid form, a solvent is preferably used for the transformation. Preferred solvents are for example alcohols, in particular ethanol or isopropanol, and water, whereby water is the most preferred solvent. The result is a dispersion or solution depending on the type of composition. A solution is preferred.

The composition may also be in the form of a solid aggregate state. Compositions in the solid aggregate state are, in the sense of the invention, understood to be compositions that are present at ambient temperatures in the solid aggregate state, and are for example powders, flakes, pellets, granulates or plates that can be transported and stored in this form without a problem.

Under another aspect the present invention relates to a composition comprising a comb polymer, a mineral binding agent and a complex made of Fe(III) with at least one Fe(III)-complexing agent X and if necessary other complexing agents, with the Fe(III)-complexing agent X being an Fe(III)-complexing agent X as described above.

The mineral binding agent is typically a hydraulic binding agent, a latent hydraulic binding agent or a non-hydraulic binding agent, in particular cement, preferably Portland cement or its blends with fly ash, silica fume, granulated blast-furnace sands and limestone fillers.

Typically the weight fraction of the comb polymer is 0.01 to 10% by weight, preferably 0.2 to 2% by weight relative to the total weight of the mineral binding agent.

Under another aspect this invention relates to a method for stabilizing comb polymers in the presence of Fe(III), whereby at least one Fe(III)-complexing agent X is added to the comb polymers. The comb polymer and the Fe(III)-complexing agent X concerns comb polymers and Fe(III)-complexing agent X as described above.

The term “stabilization” means in particular that the comb polymers are not decomposed over a long period of time and that their effect, for example as dispersing agents, grinding agents, thickeners or cement refiners lingers. Preferably the stabilization of the comb polymer remains effective for at least one week, preferably at least 4 weeks.

Preferably the Fe(III)-complexing agent X is used in an amount ranging between 0.01 and 50% by weight, in particular between 0.05 and 20% by weight relative to the total weight of the comb polymer.

It is also advantageous if in addition at least one antioxidant is added. Compounds described above as being suitable as antioxidants are suitable as antioxidants.

Preferably the at least one antioxidant is used in an amount ranging between 0.01 and 50% by weight, in particular between 0.05 and 10% by weight relative to the total weight of the comb polymer.

Preferably the method is a method at a temperature of at least 40° C., in particular 80-160° C.

Preferably the method is a method for stabilizing polymers in the presence of Fe(III) in a mineral binding agent. Compounds described above as being suitable are suitable as a mineral binding agent.

For example cement clinkers, after a combustion process exceeding 1000° C., are usually cooled to a temperature between about 100 and 200° C. and normally stored at a temperature between 80 and 150° C., in particular at about 80 to 120° C., for example in silos. Thus temperatures of at least 40° C., preferably between 80 and 160° C., may occur with this method during grinding, in particular when grinding cement clinkers to cement, and furthermore also during the subsequent storage and/or transportation of the mineral binding agent, in particular when the storage, transportation or packing environment are heated by the sun. This is therefore preferably a method for stabilizing comb polymers in the presence of Fe(III) in mineral binding agents and at a temperature of at least 40° C., preferably 80-160° C., whereby this temperature may arise during the grinding of the mineral binding agent and/or the subsequent storage.

The comb polymer and/or the Fe(III)-complexing agent X and/or the antioxidants may be added to the mineral binding agent simultaneously or at different times.

The addition of the comb polymers and/or the Fe(III)-complexing agent X and/or the antioxidants to the mineral binding agent may be applied to the mineral binding agents during the conveyance of the mineral binding agent, in particular when transporting cement. Preferably the comb polymers and/or the Fe(III)-complexing agents X and/or the antioxidants are added to the mineral binding agent, in particular cement, during the transportation process, for example in transportation channels, to the storage place, for example to the silo or to means of transport such as e.g. a truck.

The comb polymers and/or the Fe III)-complexing agent X and/or the antioxidants may be added to the mineral binding agent also before grinding the mineral binding agent, in particular before and/or during the grinding of the cement clinker.

In a preferred embodiment the Fe(III)-complexing agent X is blended beforehand with the comb polymer (and if necessary with an antioxidant) and this blend is then mixed with the mineral binding agent at the desirable moment during the manufacturing process of the mineral binding agent, in particular cement, for example before the grinding, or when transporting the mineral binding agent before storage, or applied to the mineral binding agent, resulting for example in a mineral binding agent that is coated with a mixture of Fe(III)-complexing agent X and comb polymer (and possibly antioxidants). The mineral binding agent may be conveyed or transported by way of equipment known to the person skilled in the art, for example via air conveyer chutes in which transportation take place pneumatically and gravimetrically. The grinding process usually takes place in a cement mill, for example in a ball mill. However in principle other mills known in the cement industry may also be used.

In a further aspect the present invention relates to the use of a Fe(III)-complexing agent X for stabilizing comb polymers in the presence of Fe(III).

The comb polymer and the Fe(III)-complexing agent X are the comb polymers and Fe(III)-complexing agents X as described above.

### EXAMPLES

The invention will now be explained in more detail using examples.

1. Raw Materials Used

TABLE 1
Raw materials used

| No. | Type | Manufacturer |
|---|---|---|
| KB-1 | 2-aminothiazole | Fluka, Switzerland |
| KB-2 | Benzylthiocyanate | Fluka, Switzerland |
| KB-3 | Ethylenediamine tetraacetic | Fluka, Switzerland |
| | acid (EDTA) | |
| AntO | Sterically hindered phenol | |
| F2S | Iron(II)sulfate heptahydrate | Merck Chemicals, Deutschland |
| F3S | Iron(III)sulfate hydrate | Fluka, Switzerland |
| NaSCN | Sodiumthiocyanate | Fluka, Switzerland |

2. Mortar Tests

The effectiveness of the compositions according to the invention was tested in mortar.

In a first step Portland cement (Swiss CEM I 42.5R) was coated, by blending in a kitchen mixer (Moulinex), with 1% by weight comb polymer (Sika® ViscoCrete® 110 CH, available from Sika Switzerland AG) relative to the weight of the Portland cement, and if necessary also with an amount indicated in table 2 (relative to the total weight of the comb polymer) of an Fe(III)-complexing agent X (KB), an antioxidant (AntO) or an Fe(III)-complexing agent X as well as an antioxidant. The cement coated in this way was used either immediately or stored in a transport container at 60° C. for the duration indicated in table 2 before being used in the production of mortar blends.

The following mortar mixture was produced from the coated cement:

| Composition of the mortar blend: | |
|---|---|
| (Maximum particle size 8 mm) | Quantity in g |
| Coated Portland cement | 750 |
| (Swiss CEM II/A-LL 42.5N) | |
| Limestone filler | 141 |
| Sand 0-1 mm | 738 |
| Sand 1-4 mm | 1107 |
| Sand 4-8 mm | 1154 |

The sands, the filler and the coated cement were dry-mixed for 1 minute in a Hobart mixer. The tempering water was added within 30 seconds and then mixed further for 2.5 minutes. The water/cement value (w/c value) was 0.46. In order to determine the effectiveness, the slump (ABM) (table 2) of the mortar was determined after 0 minutes following EN 1015-3. The determination of the slump that was used differs from EN 1015-3 in that the slump table was not lifted and dropped.

The examples 1 to 6 represent examples according to the invention, while the examples 7 to 12 are comparison examples.

TABLE 2
Slump (ABM) of a mortar blend in mm after 0 minutes (min)
when the coated cement is used immediately or when the
coated cement is stored at 60° C. for 2, 3 and 7 days (d).

| | | % of additives | | ABM |
|---|---|---|---|---|
| | | by weight, relative to | Days (d) after | (mm) |
| No. | Additives | the comb polymer | storage 60° C. | 0 min |
| 1 | KB 1 | 8% by weight | 3 | 226 |
| 2 | KB-1 | 8% by weight | 7 | 143 |
| 3 | KB-2 | 8% by weight | 2 | 226 |
| 4 | KB-3 | 10% by weight | 3 | 108 |
| 5 | KB-1, | 5% by weight (KB-1), | 3 | 227 |
| | AntO | 0.5% by weight (AntO) | | |
| 6 | KB-1, | 5% by weight (KB-1), | 7 | 225 |
| | AntO | 0.5 by weight (AntO) | | |
| 7 | — | — | used immediately | 227 |
| 8 | — | — | 2 | 100 |
| 9 | — | — | 3 | 100 |
| 10 | — | — | 7 | 100 |
| 11 | AntO | 0.5% by weight | 3 | 184 |
| 12 | AntO | 0.5% by weight | 7 | 146 |

The results in table 2 show that the compositions according to the invention are suitable to stabilize comb polymers even at higher temperatures (60° C.).

3. Chromate Reduction

The effectiveness of the chromate reduction by iron(II) sulfate heptahydrate (F2S) in the presence of 2-aminothiazole (KB-1) was tested in concrete.

20 g cement (CEM II/A-LL 42.5N), 40 g water and, as the case may be, the additives listed in table 3 were blended for 15 minutes under vigorous stirring on a magnet agitator and then filtered off through a Whatman 597½ pleated filter. The Cr(VI) content of the throughput was determined reflectrometrically by means of a chromate test by Merck, Germany, following the instructions of the manufacturer.

TABLE 3
CR(VI) content in ppm of the filtered throughput.

| | | % of additives by weight, relative | Conc. |
|---|---|---|---|
| No. | Additives | to the weight of the cement | Cr(VI) (ppm) |
| 13 | — | — | 5.5 |
| 14 | F2S | 0.4% by weight | 0.3 |
| 15 | KB-1 | 0.4% by weight | 5.1 |
| 16 | KB-1, F2S | 0.4% by weight (KB-1), 0.4% by | 0.2 |
| | | weight (Fe(II)sulfate) | |

Table 3 shows that the effectiveness of the Cr(VI) reduction using iron(II)sulfate heptahydrate is not impaired by the addition of 2-aminothiazole.

4. Reduction from Fe(III) to Fe(II)

40 g water and 0.2 g Fe(III)sulfate hydrate (F3S solution) and, as the case may be, 0.6 g 2-aminothiazole (KB-1) were blended for 15 minutes under vigorous stirring on a magnet agitator, the pH was set to 5 and then an iron(II) detection was carried out after 2 min and after 17 hours respectively. The iron(II) detection took place using a Reflectoquant iron(II) test by Merck, Germany, following the instructions of the manufacturer.

TABLE 4
Detection of Fe(II).

| No. | Solution | Sampling | Fe(II) detection |
|---|---|---|---|
| 17 | F3S Solution | after 2 min | below the detection limit |
| 18 | F3S Solution | after 17 h | below the detection limit |
| 19 | F3S Solution, KB-1 | after 2 min | below the detection limit |
| 20 | F3S Solution, KB-1 | after 17 h | positive detection of Fe(II) |

Table 4 shows that 2-aminothiazole caused the reduction of Fe (III) to Fe (II).

5. Selectivity

In a first test 0.2 g Fe(II)sulfate heptahydrate (F2S) was dissolved in 40 g water under vigorous stirring on a magnet agitator. Afterwards the iron(II) content was determined. In a second test 0.2 g Fe(II)sulfate heptahydrate (F2S) was dissolved in 40 g water under vigorous stirring on a magnet agitator. Afterwards 0.6 g of 2-aminothiazole (KB-1) was added. Afterwards, the iron(II) content was determined.

The iron(II) content was determined using a Reflectoquant iron(II) test by Merck, Germany following the instructions of the manufacturer.

TABLE 5
Fe(II) content in mg/l.

| No. | Solution | Additives | Conc. Fe(II) (mg/L) |
|---|---|---|---|
| 21 | F2S Lsg | — | 20 |
| 22 | F2S Lsg | KB-1 | 19 |

Table 5 shows, especially when compared with Table 4, that 2-aminothiazole barely forms any complexes with Fe (II), contrary to what happens with Fe (III), and represents therefore a selective Fe(III)-complexing agent for Fe(II).

6. Complexing

0.2 g Fe(III)sulfate hydrate (F3S) was dissolved I 40 g water under vigorous stirring on a magnet agitator and the pH was set to 5. Subsequently, in a first test 1 g sodium thiocyanate (NaSCN) was added, and after 1 min the solution was examined for a possible color change. Then 1 g 2-aminothiazole (KB-1) was added and after 1 minute the solution was again examined for a possible color change. In a second test (NaSCN) and (KB-1) were added in reverse order.

TABLE 6
Detection of Fe(II).

| | | | Color of | | Color of |
|---|---|---|---|---|---|
| No. | Solution | Additive 1 | solution | Additive 2 | solution |
| 23 | F3S solution | NaSCN | brown color | KB-1 | yellow color |
| | | | | | (change) |
| 24 | F3S solution | KB-1 | yellow color | NaSCN | yellow color |
| | | | | | (no change) |

Table 6 shows that 2-aminothiazole is suitable as an Fe(III)-complexing agent and forms stronger Fe(III)-complexes than sodium thiocyanate.

Of course, the invention is not limited to examples of the embodiments shown and described.

1. Composition comprising a comb polymer and one Fe(III)-complexing agent X selected from the group consisting of thiocyanates, condensed phosphates, amines, amino acetic acids, oxycarboxylic acids and compounds of the formula (V), whereby in the compounds of the formula (V) 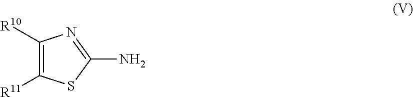
R¹⁰ and R¹¹ independently of each other stand for H, halogen atom, NO₂ or a saturated or unsaturated alkyl radical, possibly branched, with 1 to 25 C-atoms or an aryl radical with 6 to 14 C-atoms or an acyl radical with 1 to 10 C-atoms or a carbonyl radical with 1 to 10 C-atoms or a carboxyl radical with 1 to 10 C-atoms, or R¹⁰ and R¹¹ are part of an aromatic or cyclo-aliphatic ring.

2. Composition according to claim 1, wherein the Fe(III)-complexing agent X has a complexing constant for Fe(III) which is 10 times or more greater than the complexing constant for Fe(II).

3. Composition according to claim 1, wherein the Fe(III)-complexing agent X brings about a reduction of Fe(III) to Fe(II).

4. Composition according to claim 1, wherein the weight fraction of the Fe(III)-complexing agent X is 0.01 to 50% by weight relative to the total weight of the comb polymer.

5. Composition according to claim 1, wherein the composition also has one antioxidant in an amount ranging from 0.01 to 50% by weight relative to the total weight of the comb polymer.

6. Composition according to claim 5, wherein the antioxidant is selected from the group consisting of substituted phenols; substituted hydroquinones; sterically hindered aromatic amines; arylamine ketone condensation products; organo sulfuric compounds; organo phosphorus compounds; tocopherols and their derivatives; gallic acids and their derivatives and vanillin.

7. Composition comprising a comb polymer, a mineral binding agent and a complex of Fe(III) with one Fe(III)-complexing agent X and other complexing agents, whereby the Fe(III)-complexing agent X is an Fe(III)-complexing agent X whose composition is described in claim 1.

8. Composition according to claim 7, wherein the mineral binding agent is a hydraulic binding agent, a latent hydraulic binding agent or a non-hydraulic binding agent, and/or blends thereof with fly ash, silica fume, slag sands and limestone fillers.

9. Composition according to claim 7, wherein the weight fraction of the comb polymer is 0.01 to 10% by weight relative to the total weight of the mineral binding agent.

10. Method for stabilizing comb polymers in the presence of Fe(III), wherein one Fe(III)-complexing agent X is added to the comb polymers, whereby the Fe(III)-complexing agent X is an Fe(III)-complexing agent X whose composition is described in claim 1.

11. Method according to claim 10, wherein one antioxidant is added.

12. Method according to claim 10, wherein the method relates to a method at a temperature of at least 40° C.

13. Method according to claim 10, wherein the method relates to a method in a mineral binding agent.

14. Method according to claim 10, wherein the method relates to a method for stabilizing comb polymers in the presence of Fe(III) in mineral binding agents and at a temperature of at least 40° C., whereby this temperature occurs when grinding the mineral binding agent and/or during subsequent storage.

15. An Fe(III)-complexing agent X stabilizes comb polymers in the presence of Fe(III), wherein the Fe(III)-complexing agent X relates to an Fe(III)-complexing agent X whose
composition is described in claim 1.
